(19) United States
(12) Patent Application Publication
(10) Pub. No.: US 2023/0257655 A1
(43) Pub. Date: Aug. 17, 2023

(54) COMPOUND WITH VERTICAL ALIGNMENT, LIQUID CRYSTAL DISPLAY PANEL, AND PREPARATION METHOD THEREOF

(71) Applicant: TCL CHINA STAR OPTOELECTRONICS TECHNOLOGY CO., LTD., Shenzhen (CN)

(72) Inventor: Song LAN, Shenzhen (CN)

(73) Assignee: TCL CHINA STAR OPTOELECTRONICS TECHNOLOGY CO., LTD., Shenzhen (CN)

(21) Appl. No.: 17/417,399

(22) PCT Filed: Dec. 30, 2020
(86) PCT No.: PCT/CN2020/141151

§ 371 Date: Jun. 23, 2021

(30) Foreign Application Priority Data
Dec. 4, 2020 (CN) 202011416208.3

(51) Int. Cl. C09K 19/56; G02F 1/1337

(52) U.S. Cl. CPC C09K 19/56; G02F 1/133711

(57) ABSTRACT

The present application discloses a compound with vertical alignment, a liquid crystal display panel, and a preparation method thereof. In the liquid crystal display panel of the present application, the liquid crystal material of the liquid crystal layer is a liquid crystal molecule doped with the compound with vertical alignment, thus omitting the alignment layer in the liquid crystal display panel or the polymerizable monomer in the liquid crystal material, and having the advantages of simplifying the preparation process, reducing production cost, and improving product yield.

### BACKGROUND

### Field of Invention

The present application relates to the display field, and particularly to a compound with vertical alignment, a liquid crystal display panel, and a preparation method thereof.

### Description of Prior Art

Liquid crystal displays (LCDs) are one of the mainstream products in the display market. They have the advantages of power savings, easy to color, thin bodies, long life, and have large-scale information display capability. Most of the liquid crystal displays on the market are backlight liquid crystal displays, including backlight module (BM) and liquid crystal display panel set on the light output side of the BM. LCD panel uses electric field to control the deflection of liquid crystal molecules to adjust the direction of light provided by the BM. so as to form different gray-scale brightness, thus producing a display picture.

Current liquid crystal display panels usually include a color filter (CF) substrate, an array substrate (AS), and a liquid crystal cell sandwiched between the color film substrate and the array substrate. Liquid crystals are a kind of liquid crystal, and the alignment of liquid crystal molecules naturally dropped into the liquid crystal cell is disordered. Therefore, it is necessary to set an alignment layer on the color film substrate and the array substrate respectively, so that the liquid crystal molecules are arranged in a specific direction. The material of the alignment layer is usually polyimide (PI).

### Description of Prior Art

First, the current alignment layer is mainly divided into friction matching PI material and optical matching PI material. Among them, friction matching PI material has the disadvantages of easy static residue, easy to generate brush marks on the surface, dust particles in the process of matching, etc. And the optical distribution PI material has the disadvantages of poor heat resistance and aging resistance, weak molecular capacity of anchoring liquid crystal. Thus, the quality of liquid crystal display panel is affected. Second, because of high polarity and high water absorption. PI is easy to change in the process of storage and transportation, resulting in nonuniform liquid crystal alignment. Third, the price of PI material is expensive, and the film forming process of PI material on the liquid crystal display panel is also more complex, which increases the manufacturing cost of the display panel. Fourth, because the PI material is usually dissolved in N-methylpyrrolidone (NMP) solvent to prepare PI solution, and then PI solution is used to prepare the alignment layer, the whole process has the disadvantages of high energy consumption, environmental protection and easy to cause harm to human body. Fifth, the uniformity and stickiness of the alignment layer and the foreign matter on the surface will affect the yield of the display panel.

### SUMMARY

The present application provides a compound with vertical alignment, a liquid crystal display panel, and a preparation method thereof, so as to solve the problems of cumbersome preparation process of alignment layer of current liquid crystal display panel, non environmental protection, high cost, unsatisfactory performance, etc.

In the first aspect, the present application provides a compound with vertical alignment, having a structure as shown in general formula (1):

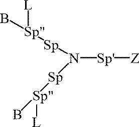

in the general formula (1), group Z is selected from a hydroxyl group or a carboxyl group;
group Sp′ is a divalent alkyl;
N is nitrogen;
group Sp refers to -(CH₂)r-Rs-(CH₂)t-, wherein values of r, s, and t ranges from 0 to 8 respectively and are not 0 at a same time, group R is any one selected from —O—, —S—, —CO—, —CO—O—, —O—CO—, —O—CO—O—, —O—CH₂—, —CH₂—O—, —CH═CH—, —C═C—, —CF═CF—, —CH═CH—CO—O—, and —O—CO—CH═CH—;
group Sp″ refers to -Xp-Yq-, wherein values of p and q ranges from 0 to 4 respectively and are not 0 at the same time, wherein X and Y are independently selected from cycloalkanes, benzene rings, or benzene rings having one or more substituents, wherein the substituent is selected from —F, —Cl, —Br, —I, —CN, —NO₂, and —C(═O)H;
the group E is selected from a straight or branched monovalent alkyl group having 5 to 20 carbon atoms, or a first group obtained by substituting at least one hydrogen atom in the monovalent alkyl group with a fluorine atom or a chlorine atom, or a second group obtained by substituting at least one —CH₂— in the monovalent alkyl group with —CO—NH—, —CO—O—, —O—CO, —S—, —CO—, or —CH═CH—, or a third group obtained by substituting at least one hydrogen atom in the second group with a fluorine atom or a chlorine atom; and
the group L is selected from the polymerizable group.

In some embodiments of the present application, in the group Sp of the general formula (1), the sum of r, s, and t is not greater than 8.

In some embodiments of the present application, the group Sp′ in the general formula (1) refers to -(CH₂)ₓ-, wherein x is a positive integer.

In some embodiments of the present application, in the group SP” of the general formula (1), the sum of p and q is not greater than 4.

In some embodiments of the present application, the group L of the general formula (1) is selected from groups containing at least one of unsaturated double bond or unsaturated triple bond.

In some embodiments of the present application, the group L is selected from one or more of the following groups:

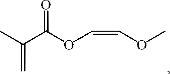

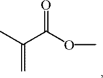

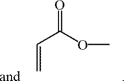

In some embodiments of the present application, the compound has any of following structural formulas:

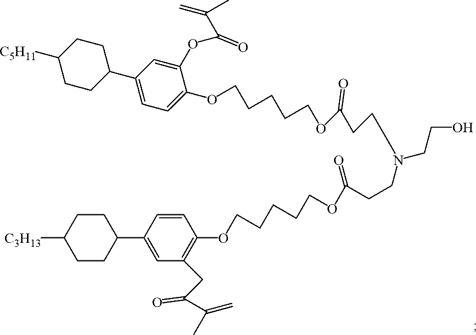

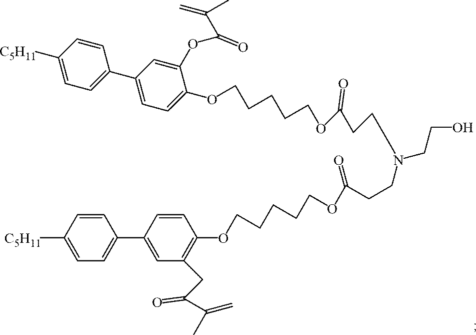

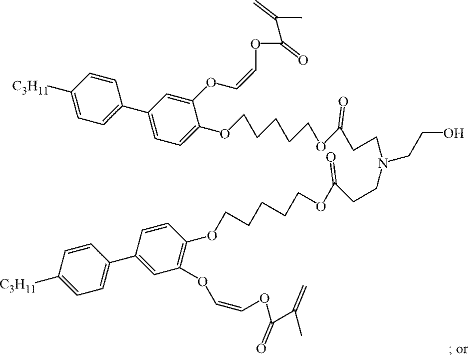

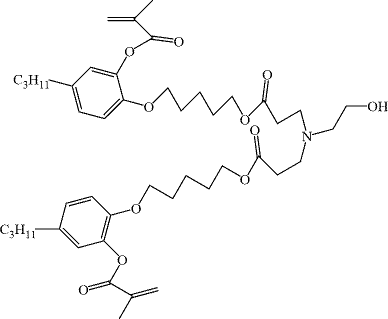

In the second aspect, the application provides a liquid crystal display panel, comprising:
a first substrate;
a second substrate disposed opposite to the first substrate ; and
a liquid crystal layer disposed between the first substrate and the second substrate,
wherein according to a total mass of the liquid crystal layer, a liquid crystal material of the liquid crystal layer comprises a compound ranging from 0.1 wt% to 5 wt% as shown in general formula (1):

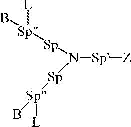

in the general formula (1), group Z is selected from a hydroxyl group or a carboxyl group;
group Sp′ is a divalent alkyl;
N is nitrogen;
group Sp refers to -(CH₂)r-Rs-(CH₂)t-, wherein values of r, s, and t ranges from 0 to 8 respectively and are not 0 at a same time, group R is any one selected from —O—, —S—, —CO—, —CO—O—, —O—CO—, —O—CO—O—, —O—CH₂—, —CH₂—O—, —CH═CH—, —C≡C—, —CF═CF—, —CH═CH—CO—O—, and —O—CO—CH═CH—;
group Sp″ refers to -Xp-Yq-, wherein values of p and q ranges from 0 to 4 respectively and are not 0 at the same time, wherein X and Y are independently selected from cycloalkanes, benzene rings, or benzene rings having one or more substituents, wherein the substituent is selected from —F. —Cl, —Br, —I, —CN, —NO₂, and —C(═O)H;
group E is selected from a straight or branched monovalent alkyl group having 5 to 20 carbon atoms, or a first group obtained by substituting at least one hydrogen atom in the monovalent alkyl group with a fluorine atom or a chlorine atom, or a second group obtained by substituting at least one —CH₂— in the monovalent alkyl group with —CO—NH—. —CO—O—, —O—CO. —S—, —CO—, or —CH═CH—, or a third group obtained by substituting at least one hydrogen atom in the second group with a fluorine atom or a chlorine atom; and
group L is selected from the polymerizable group.

In some embodiments of the present application, in the group Sp of the general formula (1), the sum of r, s, and t is not greater than 8.

In some embodiments of the present application, wherein the group Sp′ in the general formula (1) refers to -(CH₂)x-, wherein x is a positive integer.

In some embodiments of the present application, in the group SP” of the general formula (1), the sum of p and q is not greater than 4.

In some embodiments of the present application, the group L of the general formula (1) is selected from groups containing at least one of unsaturated double bond or unsaturated triple bond.

In some embodiments of the present application, the group L is selected from one or more of the following groups:

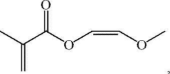

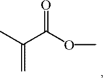

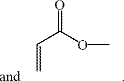

In some embodiments of the present application, the compound has any of following structural formulas:

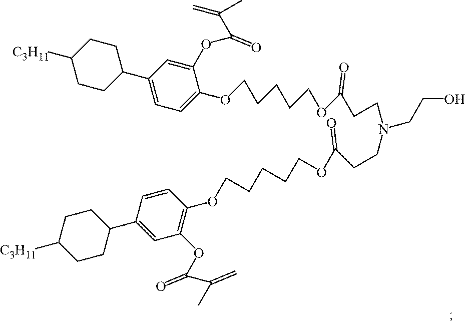

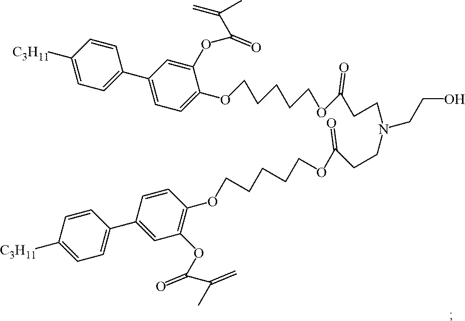

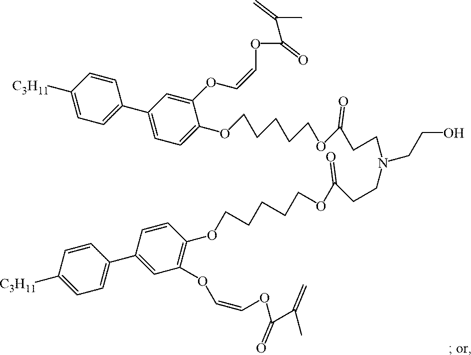

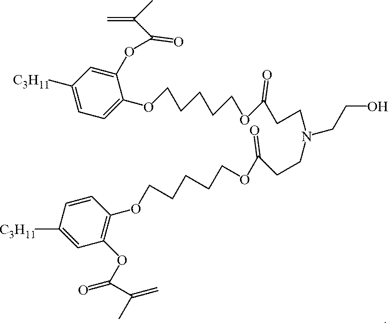

In some embodiments of the present application, wherein the first substrate is a thin film transistor array substrate, and the second substrate is a color filter substrate.

In the thired aspect, the application provides a preparation method of a liquid crystal display panel, comprising steps of:
providing a first substrate and a second substrate, and injecting a liquid crystal material between the first substrate and the second substrate, wherein according to a total mass of a liquid crystal layer, the liquid crystal material of the liquid crystal layer comprises a compound ranging from 0.1 wt% to 5 wt% as shown in general formula (1);
attaching and sealing the first substrate and the second substrate to each other to form the liquid crystal layer, wherein the compound is vertically adsorbed on surfaces of the first substrate and the second substrate by the group Z, so as to guide liquid crystal molecules to be arranged perpendicular to the first substrate and the second substrate: and
irradiating the liquid crystal layer with ultraviolet light from one side of the first substrate or the second substrate to polymerize the group L in the compound to form a polymer layer, so as to induce the liquid crystal molecules close to the surfaces of the first substrate and the second substrate to dispose with a pre-tilt angle to align the liquid crystal molecules;
wherein the general formula (1) is as follows:

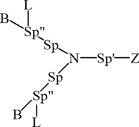

in the general formula (1), group Z is selected from a hydroxyl group or a carboxyl group;
group Sp′ is a divalent alkyl;
N is nitrogen;
group Sp refers to -(CH₂)r-Rs-(CH₂)t-, wherein values of r, s, and t ranges from 0 to 8 respectively and are not 0 at a same time, the group R is any one selected from —O—, —S—, —CO—, —CO—O—, —O—CO—. —O—CO—O—, —O—CH₂—, —CH₂—O—, —CH═CH—. —C═C—, —CF═CF—, —CH═CH—CO—O—, and —O—CO—CH═CH—;
group Sp″ refers to -Xp-Yq-, wherein values of p and q ranges from 0 to 4 respectively and are not 0 at the same time, wherein X and Y are independently selected from cycloalkanes, benzene rings, or benzene rings having one or more substituents, wherein the substituent is selected from —F, —Cl, —Br, —I, —CN, —NO₂, and —C(═O)H;
group E is selected from a straight or branched monovalent alkyl group having 5 to 20 carbon atoms, or a first group obtained by substituting at least one hydrogen atom in the monovalent alkyl group with a fluorine atom or a chlorine atom, or a second group obtained by substituting at least one —CH₂— in the monovalent alkyl group with —CO—NH—, —CO—O—, —O—CO, —S—. —CO—, or —CH═CH—, or a third group obtained by substituting at least one hydrogen atom in the second group with a fluorine atom or a chlorine atom; and
group L is selected from the polymerizable group.

The present application provides a compound with vertical alignment. Liquid crystal display panel and preparation method thereof. The compound with vertical alignment can replace the alignment layer (i.e. polyimide alignment film) in the traditional liquid crystal display panel to align the liquid crystal molecules through the photopolymerization reaction initiated by light. In the liquid crystal display panel of the present application, the liquid crystal material of the liquid crystal layer is a liquid crystal molecule doped with the compound with vertical alignment. Compared with the current twisted nematic (TN) type liquid crystal display panel, the liquid crystal display panel does not need to set an additional alignment layer, thus omitting the process of alignment layer. Compared with the current polymer stabilized vertically aligned (PSVA) liquid crystal display panel, the polymerizable monomer in the liquid crystal material is omitted, and a UV irradiation process is saved, which has the advantages of simplifying the preparation process of liquid crystal display panel, reducing production cost and improving product yield. The liquid crystal display panel prepared by the preparation method of the present application has the advantage of ideal alignment effect.

### BRIEF DESCRIPTION OF DRAWINGS

FIG. 1 is a structural schematic diagram of a liquid crystal display panel before photo alignment in an embodiment of the present application.

FIG. 2 is a structural schematic diagram of the liquid crystal display panel after photo alignment in the embodiment of the present application.

FIG. 3 is an arrangement schematic diagram of the compound (I) with vertical alignment arranged on a surface of the first (second) substrate before photo alignment in the embodiment of the present application.

FIG. 4 is an arrangement schematic diagram of the compound (I) with vertical alignment arranged on the surface of the first (second) substrate after photo alignment in the embodiment of the present application.

FIG. 5 is a schematic diagram of a preparation flow of the liquid crystal display panel in the embodiment of the present application.

FIG. 6 is an H1 nuclear magnetic resonance (NMR) spectrum of compound (I) in the embodiment of the present application.

FIG. 7 is an H1-NMR spectrum of compound (II) in the embodiment of the present application.

FIG. 8 is an H1-NMR spectrum of compound (IV) in the embodiment of the present application.

FIG. 9 is an effect diagram of the liquid crystal display panel after light leakage test in the experimental example of the present application.

FIG. 10 is an effect diagram of the liquid crystal display panel after light leakage test in the comparative example of the present application.

### DETAILED DESCRIPTION OF PREFERRED EMBODIMENTS

In order to make the above objects, features and advantages of the present invention more obvious and easy to understand, the following is a detailed description of the preferred embodiments of the present invention and the accompanying drawings. Furthermore, the directional expressions mentioned in the present invention, such as “up”, “down”, “front”, “back”, “left”, “right”, “inside”, “outside”, “side”, etc., are only referring to the direction of the additional schema. Therefore, the directional terms are used to explain and understand the invention rather than to limit the invention.

As used in the present application, “polymerizable group” refers to a group that can undergo a polymerization reaction. The polymerization reaction can be an addition polymerization reaction or a polycondensation reaction. The polymerizable group contains unsaturated bonds, which can be carbon-carbon double bond or carbon-carbon triple bond. In the embodiment of the present application, the group L is a polymerizable group, which can undergo polymerization under ultraviolet light.

In the first aspect, the embodiment of the present application provides a compound with vertical alignment, which can be applied to the liquid crystal display panel to align the liquid crystal molecules, thus omitting the alignment layer in the liquid crystal display panel or the polymerizable monomer in the liquid crystal material, which is conducive to reducing the manufacturing cost of the liquid crystal display panel and improving the yield of the liquid crystal display panel.

The compound with vertical alignment is a small molecule compound capable of photopolymerization and has a structure as shown in general formula (1):

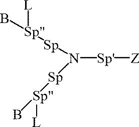

Specifically, the group Z is a head group, which is a polar group. The group SP”, group N, group Sp and group Sp″ are intermediate group. And the group E is tail group. The group L is a side group, which is polymerizable. Under the condition of UV irradiation, the adjacent molecules can polymerize to form a polymer layer.

The group Z is preferably a hydroxyl group or a carboxyl group.

The group SP’ is selected from a divalent alkyl, which may be a divalent straightr alkyl group or a divalent branched alkyl group. The preferred group SP’ is - -(CH₂)ₓ-, wherein x is a positive integer.

N is nitrogen.

The group Sp refers to -(CH₂)r-Rs-(CH₂)t-, wherein values of r, s, and t ranges from 0 to 8 respectively and are not 0 at a same time, the group R is any one selected from —O—, —S—, —CO—, —CO—O—, —O—CO—, —O—CO—O—, —O—CH₂—, —CH₂—O—, —CH═CH—, —C═C—, —CF═CF—, —CH═CH—CO—O—, and —O—CO—CH═CH—. It is preferred that the sum of r, s, and t is not greater than 8.

The group Sp″ refers to -Xp-Yq-, wherein values of p and q ranges from 0 to 4 respectively and are not 0 at the same time, wherein X and Y are independently selected from cycloalkanes, benzene rings, or benzene rings having one or more substituents, wherein the substituent is selected from —F, —Cl, —Br, —I, —CN, —NO₂, and —C(═O)H. It is preferred that the sum of p and q is not greater than 4.

The group E is selected from a straight or branched monovalent alkyl group having 5 to 20 carbon atoms, or a first group obtained by substituting at least one hydrogen atom in the monovalent alkyl group with a fluorine atom or a chlorine atom, or a second group obtained by substituting at least one —CH₂— in the monovalent alkyl group with —CO—NH—, —CO—O—, —O—CO, —S—, —CO—, or —CH═CH—, or a third group obtained by substituting at least one hydrogen atom in the second group with a fluorine atom or a chlorine atom. The preferred group E is a straight alkyl group with five carbon atoms.

The group L is selected from the polymerizable group, and it is preferred that the group L contains at least one of unsaturated double bond and unsaturated triple bond. More preferably, the group L is selected from one or more of the following groups:

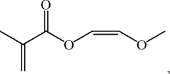

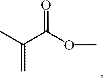

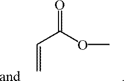

Preferably, the compound with vertical alignment is any one of the following compound (I) to compound (IV):

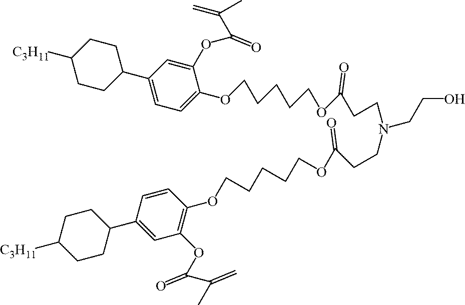

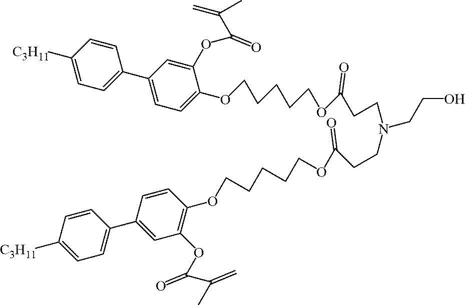

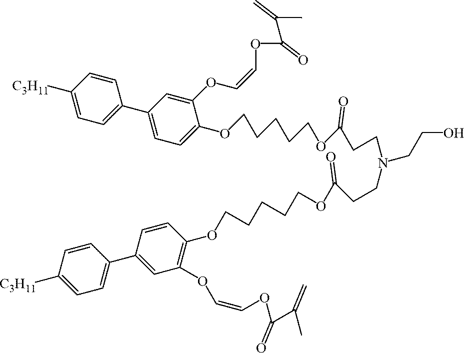

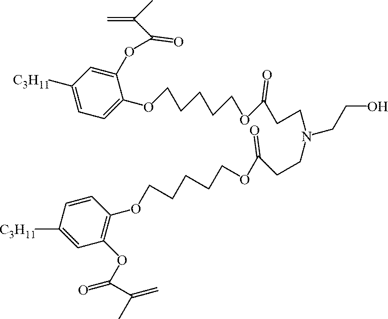

The tail group E in the compounds (I) to (IV) is a straight chain alkyl group with five carbon atoms.

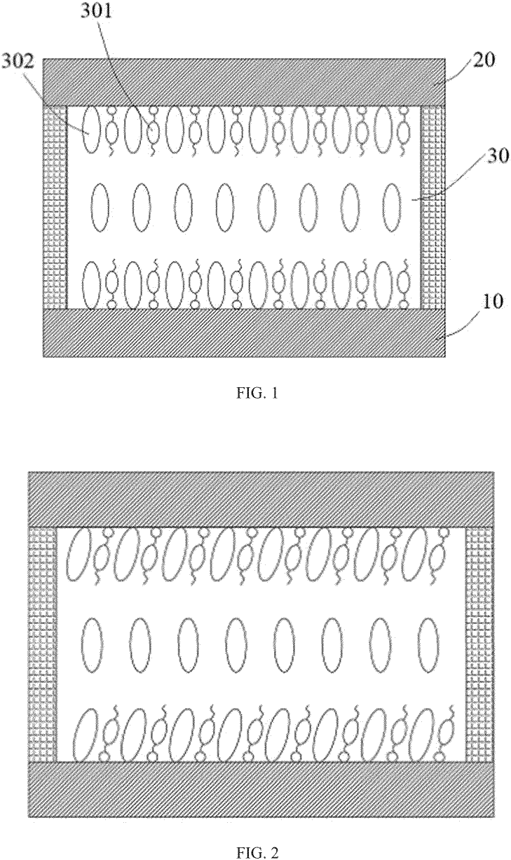

In the second aspect, the embodiments of the present application provides a liquid crystal display panel, as shown in FIG. 1 and FIG. 2. The liquid crystal display panel mainly includes a first substrate 10, a second substrate 20 and a liquid crystal layer 30. Wherein the first substrate 10 and the second substrate 20 are relatively arranged. The liquid crystal layer 30 is sandwiched between the first substrate 10 and the second substrate 20.

In one embodiment of the present application, the first substrate 10 is a thin film transistor (TFT) array substrate, and the second substrate 20 is a color filter (CF) substrate.

A first electrode layer is arranged on the surface of the first substrate 10 close to the liquid crystal layer 30. A second electrode layer is arranged on the surface of the second substrate 20 close to the liquid crystal layer 30. The first electrode layer is a common electrode, and the second electrode layer is a pixel electrode. The purpose of display is achieved by applying different voltage between the first electrode layer and the second electrode layer to drive the liquid crystal molecule deflection in the liquid crystal layer.

The first electrode layer and the second electrode layer can be metal, metal oxide, alloy or conductive non-metallic material. Wherein the metal can be copper (Cu), silver (Ag), aluminum (Al), gold (Au), platinum (Pt), chromium (Cr)., etc. The metal oxide can be indium tin oxide (In₂O₃:Sn, ITO), indium zinc oxide (ZnO:In, IZO), gallium zinc oxide (ZnO:Ga, GZO), and zinc oxide (ZnO:Al, AZO) and the like. The conductive non-metallic materials can be graphene, carbon nanotubes, etc.

According to the total mass of the liquid crystal layer, the liquid crystal material of the liquid crystal layer comprises any one of the compounds 301 with vertical alignment as described in the first aspect ranging from 0.1 wt% to 5 wt%. For example, the liquid crystal molecule 302 may be a smectic phase liquid crystal or a nematic phase liquid crystal.

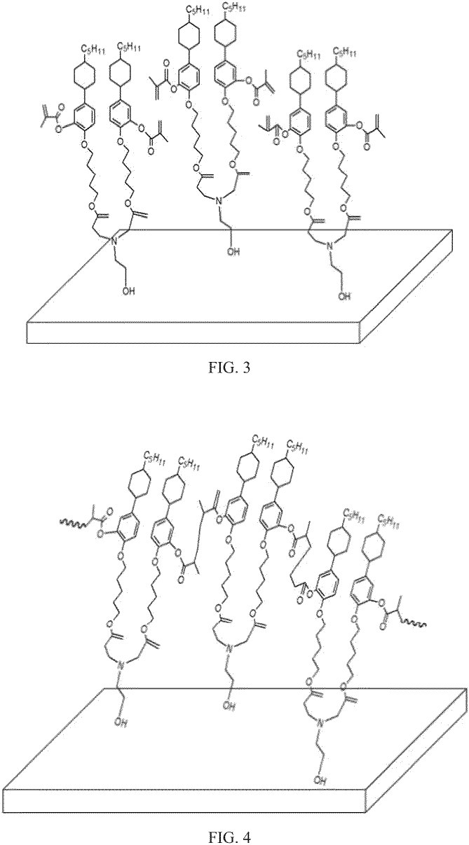

Before the liquid crystal layer 30 is irradiated by ultraviolet light, as shown in FIG. 1 and FIG. 3, the compound 301 with vertical alignment (taking compound (1) as an example) is anchored on the surface of the first substrate 10 and the second substrate 20 through the adsorption of its own head group (Group Z), and is perpendicular to the first substrate 10 and the second substrate 20. Thus, the liquid crystal molecules 302 are vertically arranged. Wherein, the group SP” and tail group (E) of the compound 301 with vertical alignment have the function of similar to the branched chain of polyimide (PI). That is, guiding the arrangement of liquid crystal molecules 302 in a stereoscopic barrier manner.

When the liquid crystal layer 30 is irradiated by ultraviolet light from an inclined direction, as shown in FIG. 2 and FIG. 4. The side group (group L) of the compound 301 with vertical alignment (taking compound (1) as an example) will undergo photopolymerization to form a polymer layer, so that the alignment direction of the whole molecule of the compound 301 with vertical alignment is changed. That is, according to the direction of ultraviolet irradiation, the compound 301 with vertical alignment is arranged on the surface of the first substrate 10 and the second substrate 20 at a certain inclination angle, thus causing the liquid crystal molecules 302 close to the surface of the first substrate 10 and the second substrate 20 to produce a pretilt angle.

In the liquid crystal display panel, the liquid crystal molecules are aligned by doping the compounds with vertical alignment into the liquid crystal molecules. Compared with the current TN type liquid crystal display panel, the liquid crystal display panel of the embodiments of the present application does not need to set additional alignment layer, thus omitting the process of alignment layer, and has the advantages of simplifying the process of liquid crystal display panel and reducing production cost. Compared with the current psva type liquid crystal display panel, the polymerizable monomer in the liquid crystal material is omitted, and a UV irradiation process is saved, which effectively reduces the production cost.

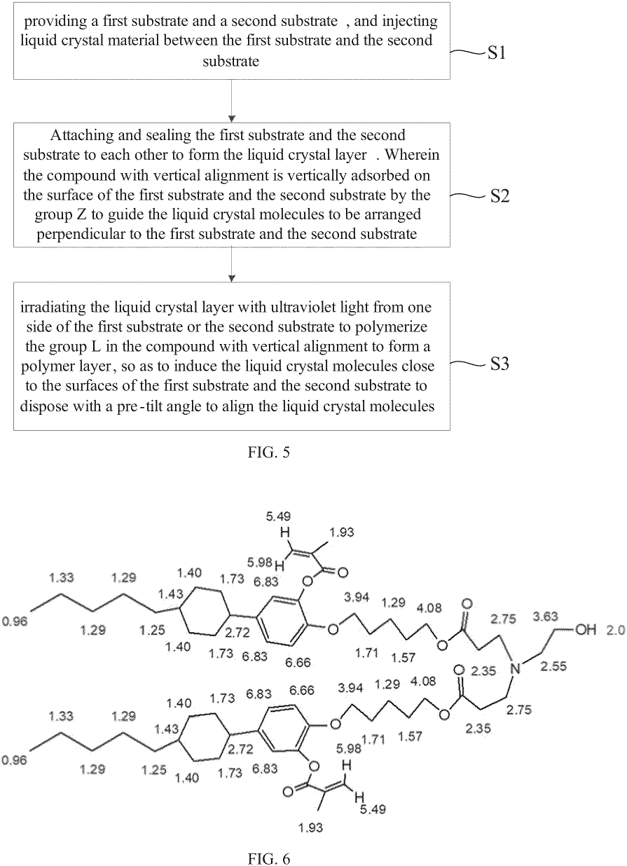

In the third aspect, the embodiment of the present application provides a method for preparing a liquid crystal display panel, which is used for preparing the liquid crystal display panel described in the second aspect. as shown in FIG. 5, and specifically includes the following steps:

S1, providing a first substrate and a second substrate, and injecting liquid crystal material between the first substrate and the second substrate.

In one embodiment of the present application, the first substrate is a TFT substrate, and the second substrate is a CF substrate. The TFT substrate and the CF substrate can be prepared by conventional technical means in the prior art, which will not be repeated here.

According to the total mass of the liquid crystal layer, the liquid crystal material of the liquid crystal layer comprises any one of the compounds 301 with vertical alignment as described in the first aspect ranging from 0.1 wt% to 5 wt%. The injection method of the liquid crystal material can adopt the conventional technical means in the prior art, such as drip injection process, etc., which will not be repeated here.

S2, Attaching and sealing the first substrate and the second substrate to each other to form the liquid crystal layer. Wherein the compound with vertical alignment is vertically adsorbed on the surface of the first substrate and the second substrate by the group Z to guide the liquid crystal molecules to be arranged perpendicular to the first substrate and the second substrate.

In one embodiment of the present application, the operation process of the opposite sealing and laminating is as follows: firstly, coating sealant around the first substrate and the second substrate; then, in a vacuum environment, bonding the first substrate and the second substrate oppositely, Finally, curing the sealant by heating or illumination.

S3, irradiating the liquid crystal layer with ultraviolet light from one side of the first substrate or the second substrate to polymerize the group L in the compound with vertical alignment to form a polymer layer, so as to induce the liquid crystal molecules close to the surfaces of the first substrate and the second substrate to dispose with a pre-tilt angle to align the liquid crystal molecules.

Specifically, the irradiation direction of the ultraviolet light has a certain inclination angle with the first substrate and the second substrate. After being irradiated by ultraviolet light from the inclined direction, the group L of the adjacent molecules in the compound with vertical alignment undergoes photopolymerization reaction, and crosslinks to form a dense network polymer layer, so that the arrangement direction of the whole molecules of the compound with vertical alignment is changed. That is, according to the irradiation direction of the ultraviolet light, the liquid crystal molecules are arranged on the surface of the first substrate and the second substrate at a certain angle, thus causing the liquid crystal molecules close to the surface of the first substrate and the second substrate to produce a pretilt angle.

In one embodiment of the present application, the wavelength of the ultraviolet light is 320-400 nm, the light intensity is 1-100 MW / cm2, and the irradiation time is 5-30 minutes.

The following describes the preparation methods of three compounds with vertical alignment through embodiments. And the compounds with vertical alignment correspond to the above compounds (I), (II), and (IV). Unless otherwise specified, the reagents and solutions used in the following examples are commercially available or can be prepared by methods known in the prior art.

### Embodiment 1: Preparation Method of Compound (I)

### 1.1, Preparation of Raw Materials for Synthesis of Compound (I).

Compound a is one of the raw materials for the synthesis of the compound (I). The compound a has a structure as shown in a structural formula (1.1):

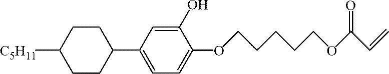

The preparation method of the compound a includes the following steps:

S1.1-1, Under the protective atmosphere of nitrogen, compound a1 and pentanediol were mixed in a molar ratio of 1:1 and reacted at room temperature (25° C.) for 72 h. The whole reaction process was carried out under the condition of ammonium chloride catalyst (5% of reactant was added to avoid aldol hydroxylation reaction), and then the reactant was extracted with n-hexane to obtain compound a2.

Specifically, the reaction formula (1.2) of step S1.1-1 is as follows:

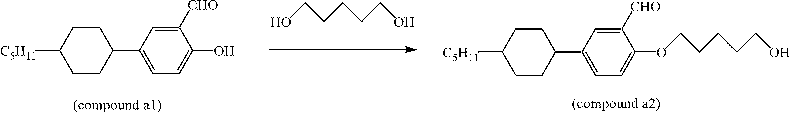

The preparation method of the compound a1 includes the following steps:

S1.1-1-1, Preparation of a compound a12. The compound a12 was prepared from a compound a11 (CAS No. 38289-29-1) with reference to Yoko Sakata, munehiro Tamiya, Masahiro Okada, et al. Switching of recognition first and reaction first mechanisms in host guest binding associated with chemical reactions [J]. Journal of the American Chemical Society, 2019.

S1.1-1-2, The compound a12 and a compound b11 (CAS No. 1761-61-1) were mixed in a molar ratio of 1:1, and then dissolved in 20 times mass of tetrahydrofuran to obtain a mixture.

S1.1-1-3, Adding potassium carbonate (K₂CO₃), tetrabutylammonium bromide (TBAB) and dichlorobis (triphenylphosphine) palladium (PD (PPh₃)₂Cl₂) to the mixture prepared in step S1.1-1-2. Wherein the mass ratio of K₂CO₃, TBAB and Pd (PPh₃)₂Cl₂ is 1:1:1, reacting at 80° C. for 8h to obtain the mixture.

S1.1-1-4, Extracting the mixture of step S1.1-1-3 with equal volume of ethyl acetate and n-hexane to obtain the Compound a1.

Specifically, the compound a12 has a structure as shown in a structural formula (1.3):

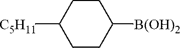

S1.1-2, Mixing the compound a2, acrylic acid and 4-(dimethylamino) pyridine in a volume ratio of 1:5:10 to obtain a mixture, and then the mixture is fully dissolved in dichloromethane (the volume ratio of the mixture to dichloromethane is 1: 10), and the whole mixing system is cooled to 1° C.

S1.1-3, Adding dichloromethane containing carbodiimide (volume ratio of carbodiimide and dichloromethane is 1:1) dropwise into the mixed system in step S1.1-2, and controlling the temperature of the mixed system at 1-4° C. during the whole dropping process. Then, stiring the reaction at room temperature (25° C.) for 24 h. filtering and collecting the filtrate, which is a compound a3.

Specifically, the compound a3 has a structure as shown in a structural formula (1.4):

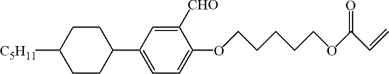

S1.1-4, The compound a3 was dissolved in a tetrahydrofuran solution containing 35% (mass%) sodium borohydride (NaBH₄), then reacted at room temperature (25° C.) for 24 h, the excess solvent was evaporated by rotation, and then extracted with methanol solution (volume ratio of water to methanol was 1:1) to obtain the compound a, wherein the mass percentage of the compound a3 in tetrahydrofuran solution was 40%.

### 1.2, Preparation of the Compound (I)

The preparation method of the compound (I) comprises the following steps:

S1.2-1, Under the protective atmosphere of nitrogen. the compound a and a compound b are mixed according to the molar ratio of 1:1 and reacted at 60° C. for 48 h. Then the reactant is extracted with the first solvent to obtain a compound c, wherein the first solvent is the solution prepared by mixing ethyl acetate and n-hexane according to the volume ratio of 1:1.

S1.2-2, Mixing the compound c, imidazole and tetrahydrofuran according to the volume ratio of 1:3:10, so that the compound c and imidazole are fully dissolved in tetrahydrofuran, and the whole mixing system is cooled to 2° C. to obtain the mixture.

S1.2-3, Adding tetrahydrofuran solution containing tert butyl dimethylchlorosilane (volume ratio of tert butyl dimethylchlorosilane to tetrahydrofuran is 1:10) dropwise to the mixture prepared in step S1.2-2, and stiring at room temperature (25° C.) for 60 min to obtain the mixture.

S1.2-4. adding 15% ammonium chloride solution by mass to the mixture prepared in step S1.2-3, and then extracting with a second solvent to obtain compound d, wherein the second solvent is methyl tert butyl ether.

S1.2-5, The compound D, methacrylic acid. 4-(dimethylamino) pyridine and dichloromethane were mixed in a molar ratio of 1:3:10:10, so that compound d, methacrylic acid and 4-(dimethylamino) pyridine were fully dissolved in dichloromethane, and the whole mixed system was cooled to 1° C. to obtain the mixture.

S1.2-6, Adding dichloromethane containing carbodiimide (volume ratio of carbodiimide and dichloromethane is 1:1) dropwise to the mixture prepared in step S1.2-5, controlling the temperature of the mixing system at 1-4° C., stiring the reaction at room temperature (25° C.) for 18 h during the whole dropping process, filtering and collecting the filtrate, and the filtrate is a compound E.

S1.2-7, The compound E is dissolved in tetrahydrofuran, and the temperature of the whole mixing system is controlled to 2° C. to obtain a mixture, wherein the mass percentage of the compound E in the mixture is 30-60%.

S1.2-8, Adding 2 mol / L hydrochloric acid dropwise to the mixture prepared in step S1.2-7, and gradually raising the temperature of the whole mixing system to room temperature (25° C.), reacting at room temperature (25° C.) for 3 h, filtering and collecting the filtrate.

S1.2-9, Carrying out H1-NMR detection on the filtrate obtained in step S8, and the detection results are shown in FIG. 6. The filtrate is the compound (I).

Specifically, the reaction formula (1.5) of step S1.2-1 is as follows:

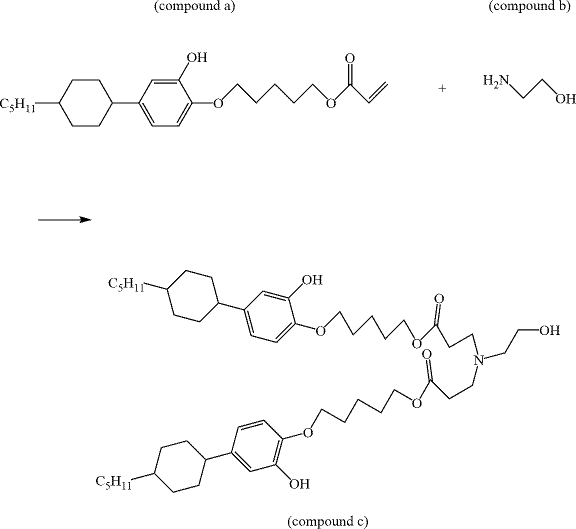

The compound d has a structure as shown in a structural formula (1.6):

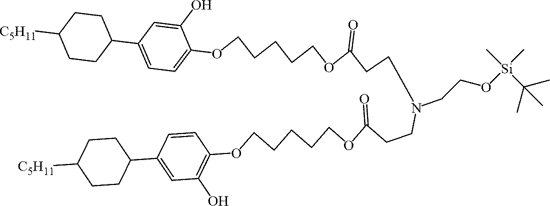

The compound E has a structure as shown in a structural formula (1.7):

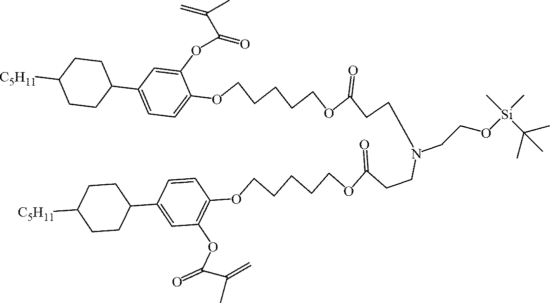

### Embodiment 2: Preparation Method of Compound (II)

### 2. 1, Preparation of Raw Materials for Synthesis of Compound (II)

Compound a′ is one of the raw materials for the synthesis of compound (II). Compound a′ has the structure as shown in a structural formula (2.1):

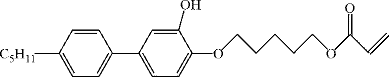

The preparation method of the compound a′ is carried out according to the preparation method of compound a in embodiment 1. It is only necessary to replace compound al with compound a1′, which has the structure as shown in a structural formula (2.2):

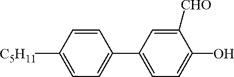

The preparation method of compound a1′ is implemented according to the preparation method of compound a1 in embodiment 1. It is only necessary to replace compound a12 with compound a12′ (CAS No. 121219-12-3), which will not be repeated here.

### 2.2, Preparation of Compound (II)

The preparation method of the compound (II) is carried out according to the steps in 1.2 of embodiment 1. It is only necessary to replace compound a with compound a′, which will not be repeated here.

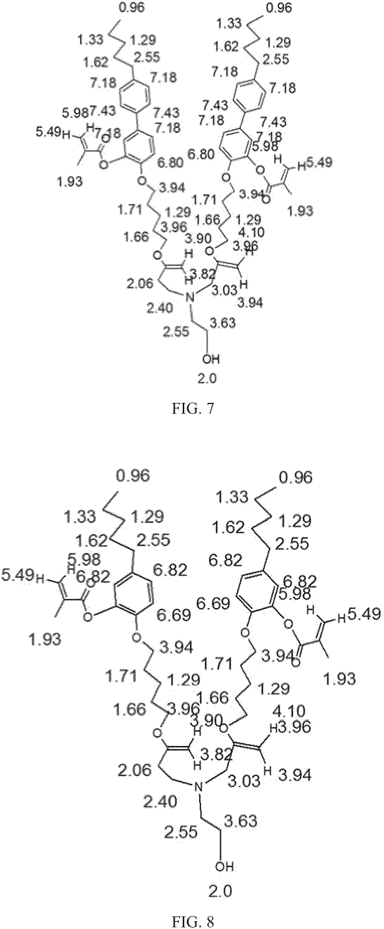

The H1 NMR spectrum of the compound (II) is shown in FIG. 7.

### Embodiment 3: Preparation Method of Compound (IV)

### 3.1, Preparation of Raw Materials for Synthesis of Compound (IV)

Compound a″ is one of the raw materials for the synthesis of compound (IV). Compound a″ has the structure as shown in a structural formula (3.1):

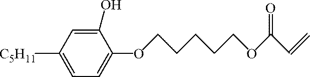

The preparation method of the compound a″ is carried out according to the preparation method of compound a in embodiment 1. It is only necessary to replace compound a1 with compound a1″, which has the structure as shown in a structural formula (3.2):

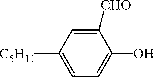

The preparation method of compound a1″ is implemented according to the preparation method of compound a1 in embodiment 1. It is only necessary to replace compound a12 with compound a12″ (CAS No. 4737-50-2), which will not be repeated here.

### 3.2, Preparation of Compound (IV)

The preparation method of the compound (IV) is carried out according to the steps in 1.2 of embodiment 1. It is only necessary to replace compound a with compound a″, which will not be repeated here.

The H1 NMR spectrum of the compound (IV) is shown in FIG. 8.

The advantages of the compounds with vertical alignment described in the embodiments of the present application are further elaborated through experimental example and comparative example.

### The Experimental Examples

This experimental example provides a liquid crystal display panel. The liquid crystal display panel mainly includes: a first substrate, a second substrate and a liquid crystal layer, wherein the first substrate and the second substrate are relatively arranged, and the liquid crystal layer is sandwiched between the first substrate and the second substrate. The first substrate is a TFT array substrate, and the second substrate is a CF substrate.

According to the mass percentage calculation, the liquid crystal material of the liquid crystal layer is composed of 1% compounds with vertical alignment and 99% liquid crystal molecules. The compound with vertical alignment are compound (I) ^{,} The liquid crystal molecule is a nematic liquid crystal.

### The Comparative Example

The comparative example provides a liquid crystal display panel, which differs from the experimental example only in that the liquid crystal material of the liquid crystal layer is not the same.

According to the mass percentage calculation, the liquid crystal material of the liquid crystal layer is composed of 1% compound (V) and 99% liquid crystal molecules. The liquid crystal molecules are nematic liquid crystals, and the compound (V) has the structure as shown in the structural formula (V):

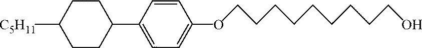

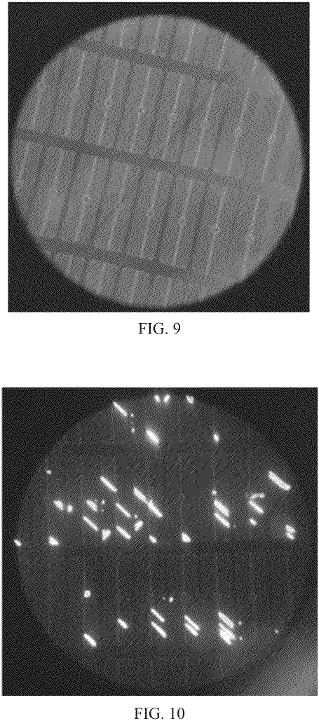

The experimental example and the proportional liquid crystal display panel are respectively tested for light leakage, and the operation flow of the light leakage test is implemented with reference to the conventional technical means in the art, which will not be repeated here. As shown in FIG. 9 and FIG. 10, the light leakage phenomenon of the proportional LCD panel is serious, while the LCD panel of the experimental example has no light leakage problem, which is proved that the alignment effect of the compound to liquid crystal molecules is ideal, which is beneficial to improve the yield of liquid crystal display panel.

The application has been described by the relevant embodiments, however, the above embodiments are only examples of the implementation of the present invention. It must be noted that the disclosed embodiments do not limit the scope of the present invention. On the contrary, the modification and equalization of the spirit and scope included in the claims are included in the scope of the invention.

What is claimed is:

1. A compound with vertical alignment, having a structure as shown in general formula (1): 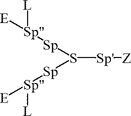
in the general formula (1), group Z is selected from a hydroxyl group or a carboxyl group;
group Sp′ is a divalent alkyl;
N is nitrogen;
group Sp refers to -(CH₂)r-Rs-(CH₂)t-, wherein values of r, s, and t ranges from 0 to 8 respectively and are not 0 at a same time, group R is any one selected from —O—, —S—, —CO—, —CO—O—, —O—CO—, —O—CO—O—, —O—CH₂—, —CH₂—O—, —CH═CH—, —C═C—, —CF═CF—, —CH═CH—CO—O—, and —O—CO—CH═CH—;
group Sp″ refers to -Xp-Yq-, wherein values of p and q ranges from 0 to 4 respectively and are not 0 at the same time, wherein X and Y are independently selected from cycloalkanes, benzene rings, or benzene rings having one or more substituents, wherein the substituent is selected from —F, —Cl, —Br, —I, —CN, —NO₂, and —C(═O)H;
group E is selected from a straight or branched monovalent alkyl group having 5 to 20 carbon atoms, or a first group obtained by substituting at least one hydrogen atom in the monovalent alkyl group with a fluorine atom or a chlorine atom, or a second group obtained by substituting at least one —CH₂— in the monovalent alkyl group with —CO—NH—, —CO—O—, —O—CO, —S—, —CO—, or —CH═CH—, or a third group obtained by substituting at least one hydrogen atom in the second group with a fluorine atom or a chlorine atom; and
group L is selected from the polymerizable group.

2. The compound of claim 1, wherein in the group Sp of the general formula (1), the sum of r, s, and t is not greater than 8.

3. The compound of claim 1, wherein the group Sp′ in the general formula (1) refers to -(CH₂)x-, wherein x is a positive integer.

4. The compound of claim 1, wherein in the group SP” of the general formula (1), the sum of p and q is not greater than 4.

5. The compound of claim 1, wherein the group L of the general formula (1) is selected from groups containing at least one of unsaturated double bond or unsaturated triple bond.

6. The compound of claim 5, wherein the group L is selected from one or more of the following groups: 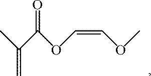 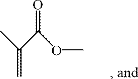 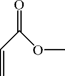
.

7. The compound of claim 1, wherein the compound has any of following structural formulas: 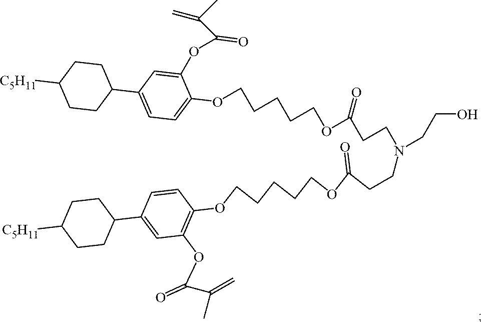 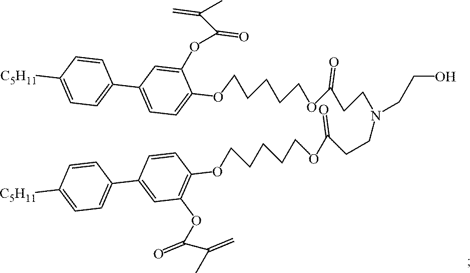 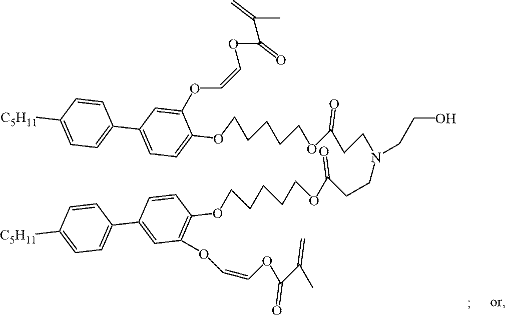 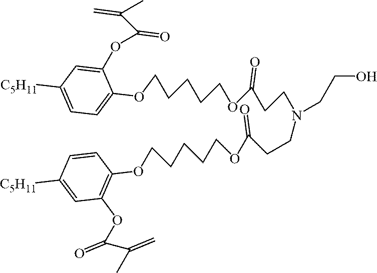
.

8. A liquid crystal display panel, comprising:
a first substrate;
a second substrate disposed opposite to the first substrate; and
a liquid crystal layer disposed between the first substrate and the second substrate,
wherein according to a total mass of the liquid crystal layer, a liquid crystal material of the liquid crystal layer comprises a compound ranging from 0.1 wt% to 5 wt% as shown in general formula (1): 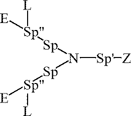
in the general formula (1), group Z is selected from a hydroxyl group or a carboxyl group;
group Sp′ is a divalent alkyl;
N is nitrogen;
group Sp refers to -(CH₂)r-Rs-(CH₂)t-, wherein values of r, s, and t ranges from 0 to 8 respectively and are not 0 at a same time, the group R is any one selected from —O—, —S—, —CO—, —CO—O—, —O—CO—, —O—CO—O—, —O—CH₂—, —CH₂—O—, —CH═CH—, —C≡C—, —CF═CF—, —CH═CH—CO—O—, and —O—CO—CH═CH—;
group Sp″ refers to -Xp-Yq-, wherein values of p and q ranges from 0 to 4 respectively and are not 0 at the same time, wherein X and Y are independently selected from cycloalkanes, benzene rings, or benzene rings having one or more substituents, wherein the substituent is selected from —F, —Cl, —Br, —I, —CN, —NO2, and —C(═O)H;
group E is selected from a straight or branched monovalent alkyl group having 5 to 20 carbon atoms, or a first group obtained by substituting at least one hydrogen atom in the monovalent alkyl group with a fluorine atom or a chlorine atom, or a second group obtained by substituting at least one —CH₂— in the monovalent alkyl group with —CO—NH—, —CO—O—, —O—CO, —S—, —CO—, or —CH═CH—, or a third group obtained by substituting at least one hydrogen atom in the second group with a fluorine atom or a chlorine atom; and
group L is selected from the polymerizable group.

9. The liquid crystal display panel of claim 8, wherein in the group Sp of the general formula (1), the sum of r, s, and t is not greater than 8.

10. Theliquid crystal display panel of claim 8, wherein the group Sp′ in the general formula (1) refers to -(CH₂)ₓ-, wherein x is a positive integer.

11. The liquid crystal display panel of claim 8, wherein in the group SP” of the general formula (1), the sum of p and q is not greater than 4.

12. The liquid crystal display panel of claim 8, wherein the group L of the general formula (1) is selected from groups containing at least one of unsaturated double bond or unsaturated triple bond.

13. The liquid crystal display panel of claim 12, wherein the group L is selected from one or more of the following groups: 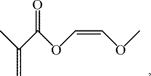 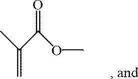 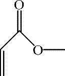
.

14. The liquid crystal display panel of claim 8, wherein the compound has any of following structural formulas: 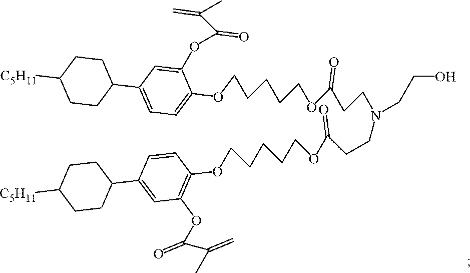 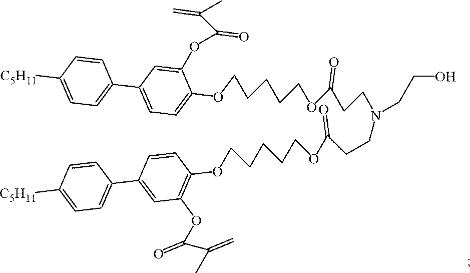 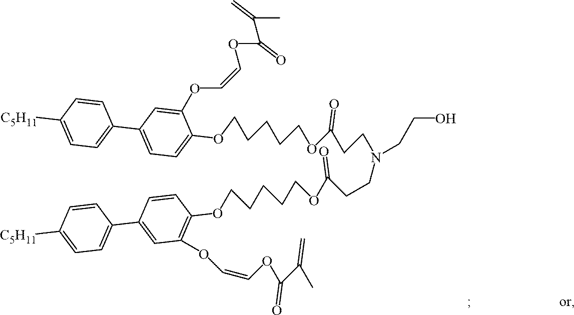 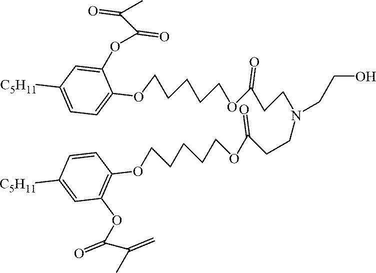
.

15. The liquid crystal display panel of claim 8, wherein the first substrate is a thin film transistor array substrate, and the second substrate is a color filter substrate.

16. A preparation method of a liquid crystal display panel, comprising steps of:
providing a first substrate and a second substrate, and injecting a liquid crystal material between the first substrate and the second substrate, wherein according to a total mass of a liquid crystal layer, the liquid crystal material of the liquid crystal layer comprises a compound ranging from 0.1 wt% to 5 wt% as shown in general formula (1);
attaching and sealing the first substrate and the second substrate to each other to form the liquid crystal layer, wherein the compound is vertically adsorbed on surfaces of the first substrate and the second substrate by the group Z, so as to guide liquid crystal molecules to be arranged perpendicular to the first substrate and the second substrate; and
irradiating the liquid crystal layer with ultraviolet light from one side of the first substrate or the second substrate to polymerize the group L in the compound to form a polymer layer, so as to induce the liquid crystal molecules close to the surfaces of the first substrate and the second substrate to dispose with a pre-tilt angle to align the liquid crystal molecules;
wherein the general formula (1) is as follows: 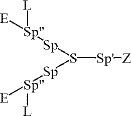
in the general formula (1), group Z is selected from a hydroxyl group or a carboxyl group;
group Sp′ is a divalent alkyl;
N is nitrogen;
group Sp refers to -(CH₂)r-Rs-(CH₂)t-, wherein values of r, s, and t ranges from 0 to 8 respectively and are not 0 at the same time, the group R is anyone selected from —O—, —S—, —CO—, —CO—O—, —O—CO—, —O—CO—O—, —O—CH₂—, —CH₂—O—, —CH═CH—, —C≡C—, —CF═CF—, —CH═CH—CO—O—, and —O—CO—CH═CH—;
group Sp″ refers to -Xp-Yq-, wherein values of p and q ranges from 0 to 4 respectively and are not 0 at the same time, wherein X and Y are independently selected from cycloalkanes, benzene rings, or benzene rings having one or more substituents, wherein the substituent is selected from —F, —Cl, —Br, —I, —CN, —NO₂, and —C(═O)H;
group E is selected from a straight or branched monovalent alkyl group having 5 to 20 carbon atoms, or a first group obtained by substituting at least one hydrogen atom in the monovalent alkyl group with a fluorine atom or a chlorine atom, or a second group obtained by substituting at least one —CH₂— in the monovalent alkyl group with —CO—NH—, —CO—O—, —O—CO, —S—, —CO—, or —CH═CH—, or a third group obtained by substituting at least one hydrogen atom in the second group with a fluorine atom or a chlorine atom; and
group L is selected from the polymerizable group.
